(19) United States
(12) Patent Application Publication
(10) Pub. No.: US 2023/0029731 A1
(43) Pub. Date: Feb. 2, 2023

(54) RING MONITOR

(71) Applicant: Cheng Uei Precision Industry Co., LTD., New Taipei City (TW)

(72) Inventor: HUNG-CHANG HUANG, New Taipei City (TW)

(21) Appl. No.: 17/827,757

(22) Filed: May 29, 2022

(30) Foreign Application Priority Data
Jul. 27, 2021 (CN) 202121724507.3

(51) Int. Cl. A61B 5/145; A61B 5/024; A61B 5/00; G16H 50/30

(52) U.S. Cl. CPC A61B 5/14542; A61B 5/02438; A61B 5/746; A61B 5/6815; G16H 50/30

(57) ABSTRACT

A ring monitor includes an accessory unit, a monitoring module, a processing unit, a transmission module and a power supply module. The accessory unit is a discontinuously annular body with a gap. Two end walls of the gap are two endpoints. The gap and the two endpoints together define an opening end. When the person who wears the ring monitor is measured, a measuring part of the person is inserted into the gap, and the measuring part of the person is clamped between the two endpoints. The monitoring module is mounted at the opening end. The processing unit is mounted in the accessory unit. The processing unit is connected with the monitoring module. The transmission module is mounted in the accessory unit. The transmission module is connected with the processing unit. The power supply module is mounted in the accessory unit.

### CROSS-REFERENCE TO RELATED APPLICATION

The present application is based on, and claims priority from, China Patent Application No. 202121724507.3, filed Jul. 27, 2021, the disclosure of which is hereby incorporated by reference herein in its entirety.

### BACKGROUND OF THE INVENTION

### 1. Field of the Invention

The present invention generally relates to a ring monitor, and more particularly to a ring monitor with a monitoring function.

### 2. The Related Art

When blood is hypoxic, maybe an invisible hypoxia is caused. The invisible hypoxia refers to a hypoxia, but a person who suffers from the invisible hypoxia is without obvious symptoms in a body of the person. A main reason of the invisible hypoxia is that an invisible hypoxia virus inhibits an alertness of a cerebral respiratory center, and a serious consequence of delaying in seeking a medical treatment. When blood oxygen is reduced and the body is without feeling the symptoms, the body has severe necrotic tissues due to a hypoxia condition. In order to detect blood oxygen conditions, most people generally use current blood oxygen machines. The current blood oxygen machine is able to be an oximeter.

However, the current blood oxygen machine proceeds with a blood oxygen detection by virtue of clamping a finger of a hand of a subject. In that case, when the current blood oxygen machine proceeds with the blood oxygen detection, the subject has no way of operating other activities which need to use the hand. As a result, an inconvenience of the current blood oxygen machine brings for the subject in use.

Thus, it is necessary to provide a ring monitor, the ring monitor is able to be worn to a person, so that when the person is proceeding with various measuring activities, the person is able to master physiological conditions at any time.

### SUMMARY OF THE INVENTION

An object of the present invention is to provide a ring monitor adapted for being cooperated with a smart device. The ring monitor is used for monitoring a person who wears the ring monitor. The ring monitor includes an accessory unit, a monitoring module, a processing unit, a transmission module and a power supply module. The accessory unit is a discontinuously annular body with a gap. Two end walls of the gap are two endpoints of the accessory unit. The gap and the two endpoints together define an opening end. When the person who wears the ring monitor is measured, a measuring part of the person is inserted into the gap, and the measuring part of the person is clamped between the two endpoints of the accessory unit. The monitoring module is mounted at the opening end. The monitoring module is mounted in the two endpoints. The monitoring module includes a blood oxygen monitoring unit for measuring a blood oxygen information of the person. The processing unit is mounted in the accessory unit. The processing unit is connected with the monitoring module for calculating the blood oxygen information measured by the monitoring module into blood oxygen parameter data. The transmission module is mounted in the accessory unit. The transmission module is connected with the processing unit for transmitting the blood oxygen parameter data to the smart device, so the blood oxygen parameter data is used in the smart device. The power supply module is mounted in the accessory unit. The power supply module is connected to the monitoring module, the processing unit and the transmission module to provide power for the monitoring module, the processing unit and the transmission module.

Another object of the present invention is to provide a ring monitor adapted for being cooperated with a smart device. The ring monitor is an annulus with a gap. The ring monitor is used for monitoring a person who wears the ring monitor. The ring monitor includes an accessory unit, two monitoring modules, a processing unit, a transmission module, a power supply module, a display unit and a warning module. Two end walls of the gap are two endpoints of the accessory unit. When the person who wears the ring monitor is measured, a measuring part of the person is inserted into the gap, and the measuring part of the person is clamped between the two endpoints of the accessory unit. The two monitoring modules are mounted in the two endpoints of the accessory unit for measuring physiological parameter information of the person. The processing unit is mounted in the accessory unit. The processing unit is connected with the two monitoring modules for calculating the physiological parameter information into a plurality of physiological parameter data. The transmission module is mounted in the accessory unit. The transmission module is connected with the processing unit. The transmission module transmits the plurality of the physiological parameter data to the smart device. The power supply module is mounted in the accessory unit. The power supply module is connected to the two monitoring modules, the processing unit and the transmission module to provide power for the two monitoring modules, the processing unit and the transmission module. The display unit is for displaying the plurality of the physiological parameter data. The warning module is connected to the display unit for providing a warning signal to warn that a physiological parameter data condition of the person is happened.

Another object of the present invention is to provide a ring monitor adapted for being cooperated with a smart device. The ring monitor is an annulus with a gap. The ring monitor is used for monitoring a person who wears the ring monitor. The ring monitor includes an accessory unit, two monitoring modules, a processing unit, a transmission module, a power supply module, a display unit, a warning module and a medical communication module. Two end walls of the gap are two endpoints of the accessory unit. When the person who wears the ring monitor is measured, a measuring part of the person is inserted into the gap, and the measuring part of the person is clamped between the two endpoints of the accessory unit. The two monitoring modules are mounted in the two endpoints of the accessory unit for measuring physiological parameter information of the person. The processing unit is mounted in the accessory unit. The processing unit is connected with the two monitoring modules for calculating the physiological parameter information into a plurality of physiological parameter data. The transmission module is mounted in the accessory unit. The transmission module is connected with the processing unit. The transmission module transmits the plurality of the physiological parameter data to the smart device. The power supply module is mounted in the accessory unit. The power supply module is connected to the two monitoring modules, the processing unit and the transmission module to provide power for the two monitoring modules, the processing unit and the transmission module. The display unit is for displaying the plurality of the physiological parameter data. The warning module is connected to the display unit for providing a warning signal to warn that a physiological parameter data condition of the person is happened. The medical communication module is connected to the warning module to send the warning signal to request a medical attention for the person.

As described above, the ring monitor is the annulus with the gap, the two endpoints of the ring monitor clamp the measuring part which is an earlobe or a front half of a nasal septum of the person, so the ring monitor continuously monitors blood oxygen concentration data and heartbeat monitoring data of the person. Moreover, when the person is proceeding with various activities, the person is able to master physiological conditions at any time.

### BRIEF DESCRIPTION OF THE DRAWINGS

The present invention will be apparent to those skilled in the art by reading the following description, with reference to the attached drawings, in which:

FIG. 1 is a perspective view of a ring monitor in accordance with a preferred embodiment of the present invention;

FIG. 2 is a system block diagram of the ring monitor in accordance with the preferred embodiment of the present invention;

FIG. 3 is another system block diagram of the ring monitor in accordance with the preferred embodiment of the present invention;

FIG. 4 is a system block diagram of a smart device, wherein the smart device is matched with the ring monitor in accordance with the preferred embodiment of the present invention; and

FIG. 5 is a schematic diagram of the smart device, wherein the smart device is matched with the ring monitor in accordance with the preferred embodiment of the present invention.

### DETAILED DESCRIPTION OF THE PREFERRED EMBODIMENT

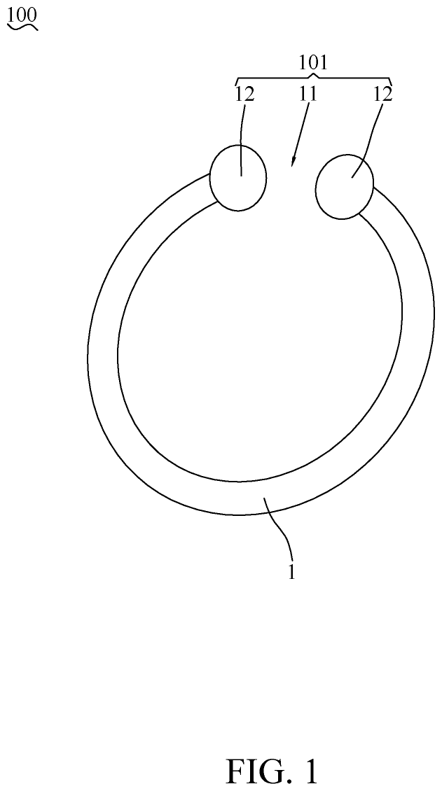

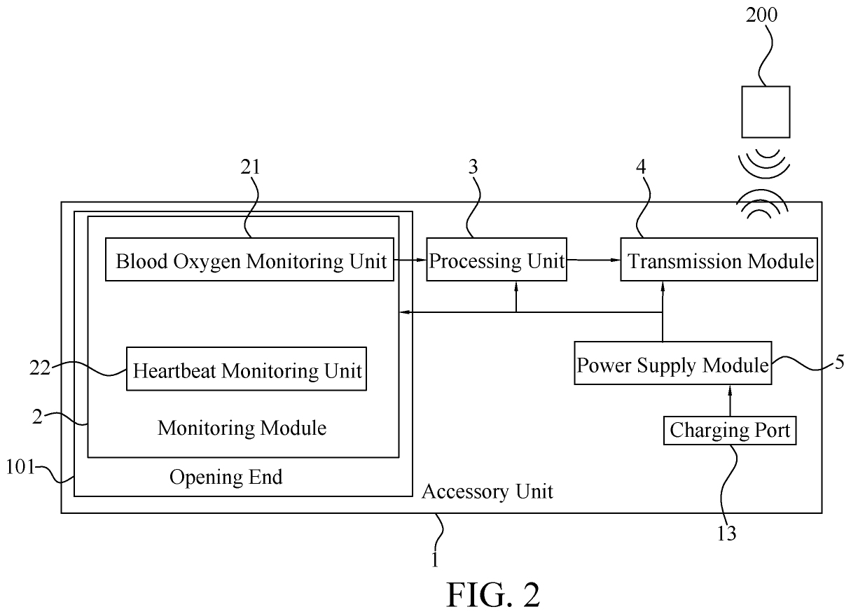

With reference to FIG. 1 and FIG. 2, a ring monitor 100 in accordance with a preferred embodiment of the present invention is shown. The ring monitor 100 is adapted for being cooperated with a smart device 200. The ring monitor 100 includes an accessory unit 1, a monitoring module 2, a processing unit 3, a transmission module 4 and a power supply module 5. The ring monitor 100 is an annulus with a gap 11. The ring monitor 100 is used for monitoring a person who wears the ring monitor 100.

The accessory unit 1 is a discontinuously annular body with the gap 11. Two end walls of the gap 11 are two endpoints 12 of the accessory unit 1. The gap 11 and the two endpoints 12 of the accessory unit 1 together define an opening end 101. The accessory unit 1 of the ring monitor 100 is an earring or a nose ring. When the person who wears the ring monitor 100 is to be measured, a measuring part 300 of a human body of the person is able to be clamped by the accessory unit 1. The measuring part 300 is able to be inserted into the gap 11, and the measuring part 300 is able to be clamped between the two endpoints 12 of the accessory unit 1.

The monitoring module 2 is mounted at the opening end 101. The monitoring module 2 is mounted in the two endpoints 12. The processing unit 3 is mounted in the accessory unit 1. The processing unit 3 is connected with the monitoring module 2 for calculating blood oxygen information measured by the monitoring module 2 into blood oxygen parameter data. The transmission module 4 is mounted in the accessory unit 1. The transmission module 4 is connected with the processing unit 3 for transmitting the blood oxygen parameter data calculated by the processing unit 3 to the smart device 200, so the blood oxygen parameter data is used in the smart device 200. The processing unit 3 is connected between the monitoring module 2 and the transmission module 4. The power supply module 5 is mounted in the accessory unit 1. The power supply module 5 is connected to the monitoring module 2, the processing unit 3 and the transmission module 4 to provide power for the monitoring module 2, the processing unit 3 and the transmission module 4.

In the preferred embodiment, the ring monitor 100 includes two monitoring modules 2. The two monitoring modules 2 are mounted in the two endpoints 12 of the accessory unit 1 for measuring physiological parameter information of the person who wears the ring monitor 100. The processing unit 3 is connected with the two monitoring modules 2 for calculating the physiological parameter information of the person into a plurality of physiological parameter data of the person. The processing unit 3 is connected with the two monitoring modules 2 for calculating the blood oxygen information into the blood oxygen parameter data. The power supply module 5 is connected to the two monitoring modules 2, the processing unit 3 and the transmission module 4 to provide the power for the two monitoring modules 2, the processing unit 3 and the transmission module 4.

In the preferred embodiment, each monitoring module 2 includes a blood oxygen monitoring unit 21 and a heartbeat monitoring unit 22. The monitoring module 2 includes the blood oxygen monitoring unit 21 and the heartbeat monitoring unit 22. The blood oxygen monitoring unit 21 is used for measuring blood oxygen information of the person who wears the ring monitor 100. The blood oxygen monitoring unit 21 is used for measuring the blood oxygen concentration data. The heartbeat monitoring unit 22 is used for monitoring heartbeats. Then the monitoring module 2 transmits monitored signals of the blood oxygen concentration data and heartbeat monitoring data to the processing unit 3. Specifically, when the person who wears the ring monitor 100 is monitored by the ring monitor 100, infrared rays and red light alternatively shine blood vessels of the person who wears the ring monitor 100, erythrocytes absorb the infrared rays and the red light, the blood oxygen concentration data is calculated through absorption differences of the infrared rays and the red light. The processing unit 3 receives the monitored signals from the monitoring module 2, and the processing unit 3 processes a signal to extract the plurality of the physiological parameter data. The plurality of the physiological parameter data includes the blood oxygen concentration data and the heartbeat monitoring data. The processing unit 3 transmits the plurality of the physiological parameter data to the transmission module 4. The transmission module 4 is able to be paired with an external device. The transmission module 4 transmits a plurality of physiological parameter data to the external device. The transmission module 4 transmits the plurality of the physiological parameter data to the smart device 200.

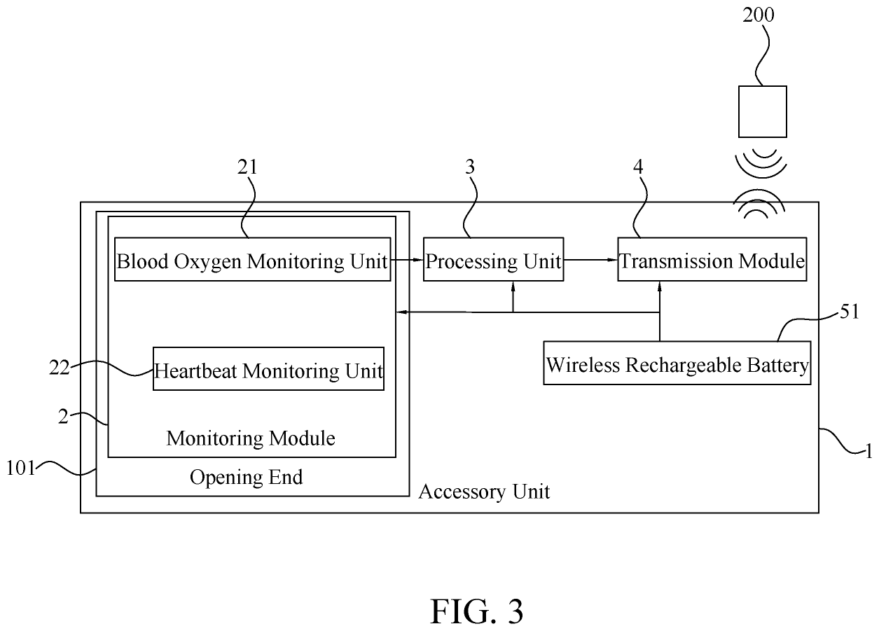

With reference to FIG. 1 to FIG. 3, the power supply module 5 is a wireless rechargeable battery 51. The ring monitor 100 further has a charging port 13. The charging port 13 is able to be connected to a charging cable to charge the wireless rechargeable battery 51. In another embodiment, the power supply module 5 is a wireless charging battery. The wireless charging battery is able to be charged by a wireless charging way, so that an operation of the power supply module 5 in the ring monitor 100 is maintained.

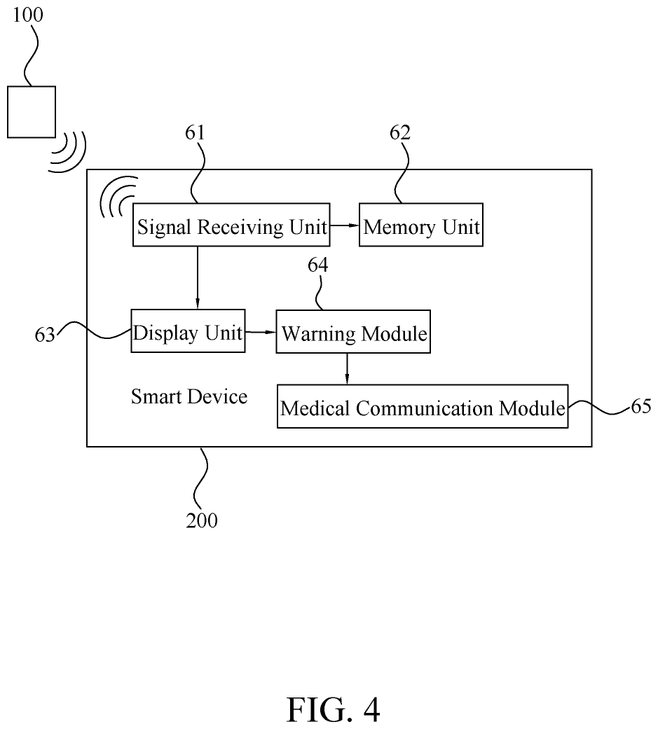

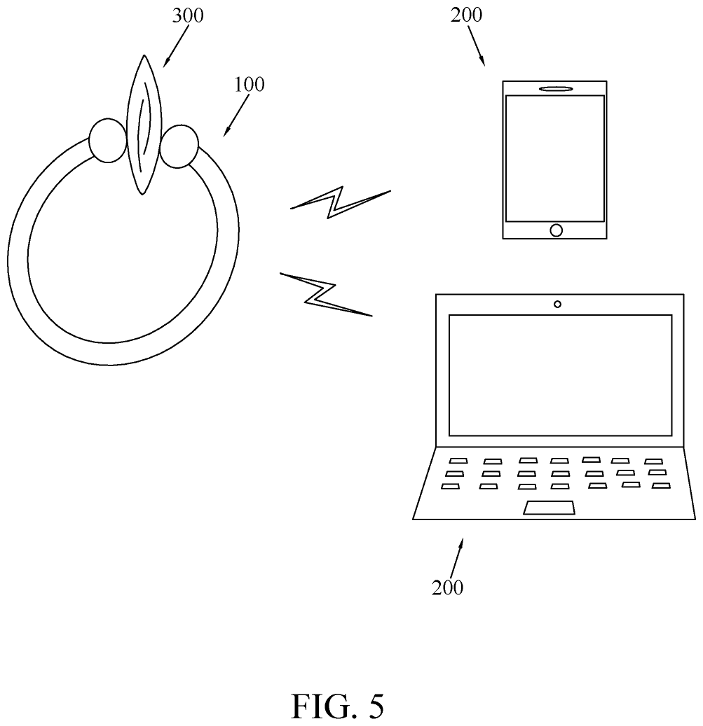

With reference to FIG. 1 to FIG. 5, in this preferred embodiment, the ring monitor 100 is corresponding to the smart device 200. The ring monitor 100 is matched with the smart device 200. The smart device 200 includes a signal receiving unit 61, at least one memory unit 62, a display unit 63, a warning module 64 and a medical communication module 65.

The signal receiving unit 61 is in communication with the transmission module 4 of the ring monitor 100. The transmission module 4 is able to transmit the plurality of the physiological parameter data to the signal receiving unit 61. Specifically, the transmission module 4 transmits data through a Bluetooth technology or a wireless network technology, and the signal receiving unit 61 receives the data through the Bluetooth technology or the wireless network technology. The signal receiving unit 61 is connected to the memory unit 62 and the display unit 63. The memory unit 62 is able to store signals in the signal receiving unit 61. The display unit 63 is used for displaying the plurality of the physiological parameter data transmitted by the transmission module 4. The display unit 63 is used for displaying the blood oxygen concentration data, the heartbeat monitoring data and other information of the plurality of the physiological parameter data received by the signal receiving unit 61.

The warning module 64 is connected to the display unit 63. The warning module 64 is used for providing a warning signal to warn that a physiological parameter data condition of the person who wears the ring monitor 100 is happened. The warning module 64 provides the warning signal to warn that a blood oxygen concentration condition or a heartbeat condition of the person who wears the ring monitor 100 is happened. The warning module 64 sets a risk value of the physiological parameter data of the person. When the monitoring module 2 monitors that the physiological parameter data of the person reaches the risk value, and the display unit 63 displays that the physiological parameter data of the person reaches the risk value, the warning module 64 determines that the person enters a dangerous state, at the moment, the warning module 64 sends out the warning signal to warn that the person need a medical attention.

The warning module 64 sets a risk value of the blood oxygen concentration data and a risk value of the heartbeat monitoring data. When the monitoring module 2 monitors that the blood oxygen concentration data or the heartbeat monitoring data reaches the risk value, and the display unit 63 displays that the blood oxygen concentration data or the heartbeat monitoring data reaches the risk value, the warning module 64 determines that the person who wears the ring monitor 100, enters the dangerous state, at the moment, the warning module 64 sends out the warning signal to warn that the person need the medical attention.

The medical communication module 65 is connected to the warning module 64 to send the warning signal to request the medical attention for the person. The medical communication module 65 is used to send the warning signal from the warning module 64 to a medical unit. When the physiological parameter data of the person reaches the risk value, the warning module 64 sends the warning signal to the medical communication module 65, then the medical communication module 65 sends the warning signal to the medical unit, so that the medical unit is able to immediately provide the medical attention for the person.

When the blood oxygen concentration data or the heartbeat monitoring data of the person reaches the risk value, the warning module 64 sends the warning signal to the medical communication module 65, then the medical communication module 65 sends the warning signal to the medical unit, so that the medical unit is able to immediately provide the medical attention for the person.

When the person who wears the ring monitor 100 is measured, the measuring part 300 of the person is inserted into the gap 11, and the measuring part 300 of the person is clamped between the two endpoints 12 of the accessory unit 1. The measuring part 300 of the person is an earlobe of the person or a front half of a nasal septum of the person. The ring monitor 100 is used as the earring or the nose ring, the earlobe of the person or the front half of the nasal septum of the person is clamped by the accessory unit 1. When the ring monitor 100 is used as the earring, the earlobe of the person is clamped between the two endpoints 12 of the accessory unit 1. When the ring monitor 100 is used as the nose ring, the front half of the nasal septum of the person is clamped between the two endpoints 12 of the accessory unit 1. Ears of the person are filled with capillaries, and the front half of the nasal septum of the person is also filled with the capillaries. The front half of the nasal septum of the person is a position where four blood vessels are converged. The two endpoints 12 of the ring monitor 100 clamp the measuring part 300 which is the earlobe or the front half of the nasal septum of the person. The ring monitor 100 monitors the blood oxygen concentration data and the heartbeat monitoring data of the capillaries in the earlobe or the front half of the nasal septum of the person by the monitoring module 2 of the opening end 101.

As described above, the ring monitor 100 is the annulus with the gap 11, the two endpoints 12 of the ring monitor 100 clamp the measuring part 300 which is the earlobe or the front half of the nasal septum of the person, so the ring monitor 100 continuously monitors the blood oxygen concentration data and the heartbeat monitoring data of the person. Moreover, when the person is proceeding with various activities, the person is able to master physiological conditions at any time.

What is claimed is:

1. A ring monitor adapted for being cooperated with a smart device, the ring monitor being used for monitoring a person who wears the ring monitor, the ring monitor comprising:
an accessory unit, the accessory unit being a discontinuously annular body with a gap, two end walls of the gap being two endpoints of the accessory unit, the gap and the two endpoints together defining an opening end, when the person who wears the ring monitor is measured, a measuring part of the person being inserted into the gap, and the measuring part of the person being clamped between the two endpoints of the accessory unit;
a monitoring module mounted at the opening end, the monitoring module being mounted in the two endpoints, the monitoring module including a blood oxygen monitoring unit for measuring a blood oxygen information of the person;
a processing unit mounted in the accessory unit, the processing unit being connected with the monitoring module for calculating the blood oxygen information measured by the monitoring module into blood oxygen parameter data;
a transmission module mounted in the accessory unit, the transmission module being connected with the processing unit for transmitting the blood oxygen parameter data to the smart device, so the blood oxygen parameter data being used in the smart device; and
a power supply module mounted in the accessory unit, the power supply module being connected to the monitoring module, the processing unit and the transmission module to provide power for the monitoring module, the processing unit and the transmission module.

2. The ring monitor as claimed in claim 1, wherein the monitoring module includes a heartbeat monitoring unit, the heartbeat monitoring unit is used for monitoring heartbeats.

3. The ring monitor as claimed in claim 1, wherein the power supply module is a wireless rechargeable battery, the ring monitor has a charging port, the charging port is able to be connected to a charging cable to charge the wireless rechargeable battery.

4. The ring monitor as claimed in claim 1, wherein the power supply module is a wireless charging battery.

5. The ring monitor as claimed in claim 1, wherein the accessory unit is an earring.

6. The ring monitor as claimed in claim 1, wherein the accessory unit is a nose ring.

7. The ring monitor as claimed in claim 1, wherein the smart device includes a display unit, the display unit is used for displaying a plurality of physiological parameter data transmitted by the transmission module.

8. The ring monitor as claimed in claim 7, wherein the smart device includes a warning module, the warning module is connected to the display unit, the warning module sets a risk value of the physiological parameter data of the person, when the monitoring module monitors that the physiological parameter data of the person reaches the risk value, and the display unit displays that the physiological parameter data of the person reaches the risk value, the warning module determines that the person enters a dangerous state, at the moment, the warning module sends out a warning signal to warn that the person need a medical attention.

9. The ring monitor as claimed in claim 8, wherein the smart device includes a medical communication module, the medical communication module is connected to the warning module, when the physiological parameter data of the person reaches the risk value, the warning module sends the warning signal to the medical communication module, then the medical communication module sends the warning signal to a medical unit, so that the medical unit is able to immediately provide the medical attention for the person.

10. The ring monitor as claimed in claim 1, wherein the measuring part of the person is an earlobe of the person, the ring monitor is used as an earring, the earlobe of the person is clamped between the two endpoints of the accessory unit.

11. The ring monitor as claimed in claim 1, wherein the measuring part of the person is a front half of a nasal septum of the person, the ring monitor is used as a nose ring, the front half of the nasal septum of the person is clamped between the two endpoints of the accessory unit.

12. A ring monitor adapted for being cooperated with a smart device, the ring monitor being an annulus with a gap, the ring monitor being used for monitoring a person who wears the ring monitor, the ring monitor comprising:
an accessory unit, two end walls of the gap being two endpoints of the accessory unit, when the person who wears the ring monitor is measured, a measuring part of the person being inserted into the gap, and the measuring part of the person being clamped between the two endpoints of the accessory unit;
two monitoring modules mounted in the two endpoints of the accessory unit for measuring physiological parameter information of the person;
a processing unit mounted in the accessory unit, the processing unit being connected with the two monitoring modules for calculating the physiological parameter information into a plurality of physiological parameter data;
a transmission module mounted in the accessory unit, the transmission module being connected with the processing unit, the transmission module transmitting the plurality of the physiological parameter data to the smart device;
a power supply module mounted in the accessory unit, the power supply module being connected to the two monitoring modules, the processing unit and the transmission module to provide power for the two monitoring modules, the processing unit and the transmission module;
a display unit for displaying the plurality of the physiological parameter data; and
a warning module connected to the display unit for providing a warning signal to warn that a physiological parameter data condition of the person is happened.

13. The ring monitor as claimed in claim 12, wherein the measuring part of the person is an earlobe of the person, the ring monitor is used as an earring, the earlobe of the person is clamped between the two endpoints of the accessory unit.

14. The ring monitor as claimed in claim 12, wherein the measuring part of the person is a front half of a nasal septum of the person, the ring monitor is used as a nose ring, the front half of the nasal septum of the person is clamped between the two endpoints of the accessory unit.

15. A ring monitor adapted for being cooperated with a smart device, the ring monitor being an annulus with a gap, the ring monitor being used for monitoring a person who wears the ring monitor, the ring monitor comprising:
an accessory unit, two end walls of the gap being two endpoints of the accessory unit, when the person who wears the ring monitor is measured, a measuring part of the person being inserted into the gap, and the measuring part of the person being clamped between the two endpoints of the accessory unit;
two monitoring modules mounted in the two endpoints of the accessory unit for measuring physiological parameter information of the person;
a processing unit mounted in the accessory unit, the processing unit being connected with the two monitoring modules for calculating the physiological parameter information into a plurality of physiological parameter data;
a transmission module mounted in the accessory unit, the transmission module being connected with the processing unit, the transmission module transmitting the plurality of the physiological parameter data to the smart device;
a power supply module mounted in the accessory unit, the power supply module being connected to the two monitoring modules, the processing unit and the transmission module to provide power for the two monitoring modules, the processing unit and the transmission module;
a display unit for displaying the plurality of the physiological parameter data;
a warning module connected to the display unit for providing a warning signal to warn that a physiological parameter data condition of the person is happened; and
a medical communication module connected to the warning module to send the warning signal to request a medical attention for the person.

16. The ring monitor as claimed in claim 15, wherein the plurality of the physiological parameter data includes blood oxygen concentration data and heartbeat monitoring data.

17. The ring monitor as claimed in claim 15, wherein the measuring part of the person is an earlobe of the person, the ring monitor is used as an earring, the earlobe of the person is clamped between the two endpoints of the accessory unit.

18. The ring monitor as claimed in claim 15, wherein the measuring part of the person is a front half of a nasal septum of the person, the ring monitor is used as a nose ring, the front half of the nasal septum of the person is clamped between the two endpoints of the accessory unit.
